(19) United States
(12) Patent Application Publication
(10) Pub. No.: US 2026/0279582 A1
(43) Pub. Date: Sep. 17, 2026

(54) ALZHEIMER'S DETECTION AND PATIENT PROTECTION

(71) Applicant: INTERNATIONAL BUSINESS MACHINES CORPORATION, Armonk, NY (US)

(72) Inventors: Yu Zhu, Beijing (CN); Su Liu, Austin, TX (US); Peng Hui Jiang, Beijing (CN); Guang Han Sui, Beijing (CN); Jun Su, Beijing (CN); Jun Feng Duan, Beijing (CN)

(21) Appl. No.: 19/079,000

(22) Filed: Mar. 13, 2025

(51) Int. Cl. G16H 50/30; G06N 20/00; G16H 10/60; G16H 50/50; G16H 50/70; G16H 80/00

(52) U.S. Cl. CPC G16H 50/30; G06N 20/00; G16H 10/60; G16H 50/50; G16H 50/70; G16H 80/00

(57) ABSTRACT

Embodiments receive user data from a device; create a user profile based on the user data; train an artificial intelligence (AI) model using the user profile and historical user profiles; determine a long term cognitive state score (CSS) and a real-time CSS using the trained AI model; determine that an Alzheimer's risk score is below a predetermined Alzheimer's threshold based on the long term CSS and the real-time CSS; and provide a notification in response to the Alzheimer's risk score being below the predetermined Alzheimer's threshold.

### BACKGROUND

Aspects of the present invention relate generally to a system and a method for Alzheimer's detection and patient protection.

Alzheimer's disease (also referred to as Alzheimer's) is a brain disease that gets worse over time and causes the brain to shrink and the brain cells to die. In particular, early symptoms of Alzheimer's include memory loss that disrupts daily life, challenges in planning or solving problems, difficulty completing familiar tasks, confusion with time or place, trouble understanding visual images and spatial relationships, new problems with words in speaking or writing, misplacing things and losing the ability to retrace steps, decreased or poor judgment, withdrawal from work or social activities, and changes in mood and personality.

### SUMMARY

In a first aspect of the invention, there is a method including: receiving user data from a device; creating a user profile based on the user data; training an artificial intelligence (AI) model using the user profile and historical user profiles; determining a long term cognitive state score (CSS) and a real-time CSS using the trained AI model; determining that an Alzheimer's risk score is below a predetermined Alzheimer's threshold based on the long term CSS and the real-time CSS; and providing a notification in response to the Alzheimer's risk score being below the predetermined Alzheimer's threshold. In particular, embodiments provide a technical improvement of determining when a user has a high Alzheimer's risk and providing a notification in response to the user having the high Alzheimer's risk.

In another aspect of the invention, there is a computer program product including one or more computer readable storage media and program instructions stored on the one or more computer readable storage media to perform operations including: receiving user data from an Internet of things (IoT) network; creating a user profile based on the user data; training an artificial intelligence (AI) model using the user profile and historical user profiles by utilizing a random forest algorithm; determining a long term cognitive state score (CSS) and a real-time CSS using the trained AI model; determining that an Alzheimer's risk score is below a predetermined Alzheimer's threshold based on the long term CSS and the real-time CSS; and providing a notification in response to the Alzheimer's risk score being below the predetermined Alzheimer's threshold. In particular, embodiments may provide a technical improvement of determining when a user has a high Alzheimer's risk and providing a notification in response to the user having the high Alzheimer's risk.

In another aspect of the invention, there is a system including a processor set, one or more computer readable storage media, and program instructions stored on the one or more computer readable storage media to cause the processor set to perform operations including: receiving user data from a wearable device; creating a user profile based on the user data; training an artificial intelligence (AI) model using the user profile and historical user profiles by utilizing a random forest algorithm; determining a long term cognitive state score (CSS) and a real-time CSS using the trained AI model; determining that an Alzheimer's risk score is below a predetermined Alzheimer's threshold based on the long term CSS and the real-time CSS; providing a caregiver notification in response to the Alzheimer's risk score being below the predetermined Alzheimer's threshold; and providing a geofence notification which dynamically adjusts a geofence for protection in response to the real-time CSS being below a real-time CSS threshold. In particular, embodiments may provide a technical improvement of determining when a user has a high Alzheimer's risk and providing a notification in response to the user having the high Alzheimer's risk.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 depicts a computing environment according to an embodiment of the present invention.

FIG. 2 shows a block diagram of an exemplary environment in accordance with aspects of the present invention.

FIG. 3 shows a block diagram of a data collection system in accordance with aspects of the present invention.

FIG. 4 shows a flowchart of an exemplary method in accordance with aspects of the present invention.

FIG. 5 shows a flowchart of an exemplary method in accordance with aspects of the present invention.

### DETAILED DESCRIPTION

Aspects of the present invention relate generally to a system and a method for Alzheimer's disease detection and patient protection. In particular, embodiments are directed to proactively detecting a cognitive state of a patient to determine whether the patient has Alzheimer's disease and to provide proactive protections for the patient in response to a determination that the patient has Alzheimer's disease.

Embodiments of the present invention proactively detect a cognitive state of a patient and take remedial action if needed. In particular, aspects of the present invention provide a system, a computer program product, and a computer-implemented method to utilize generative artificial intelligence (AI) to monitor the cognitive state of a user during a time period by analyzing user data and generating an assessment for the cognitive state of the user. In further aspects of the present invention, the system, the computer program product, and the computer-implemented method utilizes generative AI to create a user profile by using collected data from an Internet of things (IoT) network and other data sources such as a task list, calendar, etc.

Embodiments of the present invention provide a computer-implemented method, a system, and a computer program product for actively protecting patients by dynamically adjusting the environment. Aspects of the present invention prevent accidents and enhance safety in daily lives by modifying geofence settings, recognizing dangerous objects, and guiding patients through daily tasks. Embodiments of the present invention seamlessly integrate a system into a life of a patient without being intrusive by leveraging wearable technology and the IoT network. Aspects of the present invention enable continuous monitoring without significantly altering a lifestyle of a patient or requiring extensive time or financial resources.

Embodiments of the present invention integrate data from multiple sources (e.g., physical, speech, actions) to monitor early symptoms of Alzheimer's disease. Aspects of the present invention enable a detection of cognitive decline at an early stage by utilizing a multi-faceted approach. Further embodiments of the present invention create personalized profiles based on specific habits, interactions, and cognitive functions to allow for individualized care by utilizing generative AI. Further, aspects of the present invention ensure that the system adapts to the unique needs of each patient.

In a first aspect of the invention, there is a method including: receiving user data from a device; creating a user profile based on the user data; training an artificial intelligence (AI) model using the user profile and historical user profiles; determining a long term cognitive state score (CSS) and a real-time CSS using the trained AI model; determining that an Alzheimer's risk score is below a predetermined Alzheimer's threshold based on the long term CSS and the real-time CSS; and providing a notification in response to the Alzheimer's risk score being below the predetermined Alzheimer's threshold. In particular, embodiments provide a technical improvement of determining when a user has a high Alzheimer's risk and providing a notification in response to the user having the high Alzheimer's risk.

In embodiments, the creating the user profile based on the user data further includes creating the user profile based on the user data by utilizing a generative AI model, and the device includes at least one wearable device. In particular, embodiments provide a technical improvement of creating the user profile based on a generative artificial intelligence (AI) model which improves accuracy of data within the user profile.

In embodiments, the user profile includes speech patterns, interaction habits, emotional preferences, task completion, abnormal vital signs, motion patterns, unusual geospatial movements, and interactions with objects. In particular, embodiments may provide a technical improvement of the user profile including speech patterns, interaction habits, emotional preferences, task completion, abnormal vital signs, motion patterns, unusual geospatial movements, and interactions with objects which improves data within the user profile for a determination of Alzheimer's risk.

In embodiments, the AI model is trained using a random forest algorithm. In particular, embodiments may provide a technical improvement of training an AI model by utilizing a random forest algorithm which improves accuracy of determining Alzheimer's risk.

In embodiments, the long term cognitive state score (CSS) represents a cognitive state over a predetermined period of time. In particular, embodiments may provide a technical improvement of providing a long term cognitive state score (CSS) over a predetermined period of time which improves accuracy of determining Alzheimer's risk.

In embodiments, the long term cognitive state score (CSS) being determined based on plural cognitive factors, a weight assigned to each of the cognitive factors, and a normalized score for each of the cognitive factors. In particular, embodiments may provide a technical improvement of providing a long term cognitive state score being based on plural cognitive factors, a weight of the cognitive factors, and a normalized score of the cognitive factors which improves accuracy of determining Alzheimer's risk.

In embodiments, the long term cognitive state score (CSS) is determined by a formula including: long term

$$CSS = ( 1 / N ) * ( {\sum }_{i = 1}^{N} ( Wi * fi ) ) ,$$

in which N is the number of cognitive factors, Wi is the weight assigned to each cognitive factor, and fi is the normalized score for each cognitive factor. In particular, embodiments may provide a technical improvement of providing a long term cognitive state score (CSS) being based on a CSS formula which improves accuracy of determining Alzheimer's risk.

In embodiments, the Alzheimer's risk score includes a prediction of a likelihood of an onset of Alzheimer's. In particular, embodiments may provide a technical improvement of providing the Alzheimer's risk score which represents the likelihood of Alzheimer's and improves the accuracy of determining Alzheimer's risk.

In embodiments, the notification is provided to a caregiver. In particular, embodiments may provide a technical improvement of providing the notification to the caregiver which improves communication and provides quicker notification to the caregiver.

In embodiments, the notification provided to the caregiver includes a geofence notification which dynamically adjusts a geofence according to a plurality of predefined policies.

In particular, embodiments may provide a technical improvement of providing a geofence notification which dynamically adjusts the geofence according to a plurality of predefined policies improves communication to the caregiver and provides safety to an Alzheimer's patient.

In embodiments, a trend visualization is provided for cognitive health of the Alzheimer's risk score over time through an interactive graphical user interface (GUI). In particular, embodiments may provide a technical improvement of providing a trend visualization of cognitive health of the Alzheimer's risk score over time through the GUI and improves communication of a trend of Alzheimer's risk score to caregivers.

In embodiments, a determination that a user associated with the user data is using a dangerous object is based on an object detection and the real-time CSS. Embodiments may provide a technical improvement of determining that the user is using a dangerous object improves safety to an Alzheimer's patient.

In embodiments, a determination that the user associated with the user data is performing a risky action is based on an action recognition and the real-time CSS. Embodiments may provide a technical improvement of determining that the user is performing a risky action improves safety to an Alzheimer's patient.

In another aspect of the invention, there is a computer program product including one or more computer readable storage media and program instructions stored on the one or more computer readable storage media to perform operations including: receiving user data from an Internet of things (IoT) network; creating a user profile based on the user data; training an artificial intelligence (AI) model using the user profile and historical user profiles by utilizing a random forest algorithm; determining a long term cognitive state score (CSS) and a real-time CSS using the trained AI model; determining that an Alzheimer's risk score is below a predetermined Alzheimer's threshold based on the long term CSS and the real-time CSS; and providing a notification in response to the Alzheimer's risk score being below the predetermined Alzheimer's threshold. In particular, embodiments may provide a technical improvement of determining when a user has a high Alzheimer's risk and providing a notification in response to the user having the high Alzheimer's risk.

In embodiments, the user profile includes speech patterns, interaction habits, emotional preferences, task completion, abnormal vital signs, motion patterns, unusual geospatial movements, and interactions with objects. In particular, embodiments may provide a technical improvement of the user profile including speech patterns, interaction habits, emotional preferences, task completion, abnormal vital signs, motion patterns, unusual geospatial movements, and interactions with objects which improves data within the user profile for a determination of Alzheimer's risk.

In embodiments, the long term cognitive state score (CSS) represents a cognitive state over a predetermined period of time. In particular, embodiments may provide a technical improvement of providing a cognitive state score over a predetermined period of time which improves accuracy of determining Alzheimer's risk.

In embodiments, the long term cognitive state score (CSS) is determined based on plural cognitive factors, a weight assigned to each of the cognitive factors, and a normalized score for each of the cognitive factors. In particular, embodiments may provide a technical improvement of providing a long term cognitive state score being based on a number of cognitive factors, a weight of the cognitive factors, and a normalized score of the cognitive factors which improves accuracy of determining Alzheimer's risk.

In embodiments, the long term cognitive state score is determined by a formula including:

$$CSS = ( 1 / N ) * ( {\sum }_{i = 1}^{N} ( Wi * fi ) ) ,$$

in which N is the number of cognitive factors, Wi is the weight assigned to each cognitive factor, and fi is the normalized score for each cognitive factor. In particular, embodiments may provide a technical improvement of providing a long term cognitive state score being based on a CSS formula which improves accuracy of determining Alzheimer's risk.

In embodiments, the Alzheimer's risk score includes a prediction of a likelihood of an onset of Alzheimer's. In particular, embodiments may provide a technical improvement of providing the Alzheimer's risk score which represents the likelihood of the Alzheimer's and improves the accuracy of determining Alzheimer's risk.

In another aspect of the invention, there is a system including a processor set, one or more computer readable storage media, and program instructions stored on the one or more computer readable storage media to cause the processor set to perform operations including: receiving user data from a wearable device; creating a user profile based on the user data; training an artificial intelligence (AI) model using the user profile and historical user profiles by utilizing a random forest algorithm; determining a long term cognitive state score (CSS) and a real-time CSS using the trained AI model; determining that an Alzheimer's risk score is below a predetermined Alzheimer's threshold based on the long term CSS and the real-time CSS; providing a caregiver notification in response to the Alzheimer's risk score being below the predetermined Alzheimer's threshold; and providing a geofence notification which dynamically adjusts a geofence for protection in response to the real-time CSS being below a real-time CSS threshold. In particular, embodiments may provide a technical improvement of determining when a user has a high Alzheimer's risk and providing a notification in response to the user having the high Alzheimer's risk.

Embodiments of the present invention analyze daily data related to a user and provides early stage detection of Alzheimer's disease. In contrast, conventional systems rely on limited data, such as speech or medical imaging. Accordingly, conventional systems utilize a one-size fits all approach such as medical tests, which simply provide a diagnosis without providing any proactive solution to a patient who has been diagnosed with the Alzheimer's disease. Further, conventional systems track only speech and do not provide a comprehensive view of a cognitive state of a patient which also includes reaction times and reasoning abilities.

Embodiments of the present invention include a system, method, and computer program product for proactively detecting a cognitive state of a patient and providing alerts and notifications to protect the patient. Accordingly, implementations of the present invention provide an improvement (i.e., technical solution) to a problem arising in the technical field of Alzheimer's detection and patient protection. In particular, embodiments of the present invention detect speech, interactions, actions, and completion of daily work using an IoT network including a plurality of sensors and wearable devices. Further, embodiments of the present invention monitor a cognitive state of a user by analyzing user data and providing an assessment of the cognitive state of the user.

Implementations of the present invention are necessarily rooted in computer technology. For example, the steps of training an artificial intelligence (AI) model using the user profile and historical user profiles and determining a long term cognitive state score (CSS) and a real-time CSS using the trained AI model cannot be performed in the human mind (or with pen and paper). Training an AI model using the user profile and historical user profiles and determining a long term CSS and a real-time CSS using the trained AI model is, by definition, performed by a computer and cannot be performed in the human mind (or with a pen and paper). In further embodiments, the steps of training the AI model using a random forest algorithm are also rooted in computer technology and cannot be performed in the human mind (or with pen and paper).

Aspects of the present invention include a method, system, and computer program product for analyzing daily user data and providing early stage prediction of Alzheimer's disease by utilizing wearable devices, an IoT network, and a generative AI decision module. For example, a computer-implemented method includes: making a cognitive state assessment for a user based on retrieved data from a wearable device; determining actions for early state diagnosis of Alzheimer's disease and user protection using cognitive state assessment result and predefined policies; performing a cognitive state score within a certain time period to indicate degenerative change of the user and corresponding risk for the Alzheimer's disease; determining the current cognitive state of the user by utilizing the real-time cognitive state score; transmitting actions to wearable device and IoT network for enforcement; collecting physical data of user, such as blood pressure, body temperature, and heartbeat rate; collecting user voice data, including speech, conversation, self talking, and interactions from other people; collecting user daily life action data from sensors, such as camera, positioning, and pedometer sensor; transmitting the collected data to the AI decision module; providing accessible functions for a user and co-operate with the IoT network to enforce user protection based on an action request from the AI decision module; and collecting data from the daily life of the user, including video, audio, trace tracking, positioning and enforcing actions from the AI decision module using the IoT network (e.g., a smart home).

In further embodiments, the computer-implemented method includes setting up a user profile in a speaking habit, interaction habit, and an emotion preference using generative AI; analyzing user data to determine symptom factors for Alzheimer's disease, which include speaking ability (e.g., from speech data, sentiment, tones), memory issues (e.g., from speech data, interaction with people and action data from camera), time and space analysis (e.g., from daily life data), completion of familiar tasks (e.g., from daily life data, e.g., folk dropping), combine the data from other data sources (e.g., calendar or task list) to analyze the problem solving ability; generating cognitive state assessments based on collected data and symptoms of Alzheimer's disease; determining the cognitive state score within a time period to indicate degenerative change of user and corresponding risk for Alzheimer's disease; and determining the real-time cognitive state score to indicate the current cognitive state of the user. The computer-implemented method further comprises predict the likelihood of Alzheimer's disease which includes notifying the user to take medical tests in response to the cognitive state score exceeds the threshold of likelihood; and showing data and the data trend on the user interface (UI) to the user. Embodiments of the computer-implemented method further comprises determining the cognitive state of the user and communicating with a wearable device and the IoT network for proactive protections including dynamically adjusting the geofence setting based on the cognitive state score; setting up object recognition and action recognition to prevent risky action of the user (e.g., using a knife, having gas leaked, etc.); enabling accessible mode of the wearable device to give tip to a user related to the time, the space, the item, or the action in a visual or audible manner; and sending notifications to contacts.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer-readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as Alzheimer's detection code of block 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

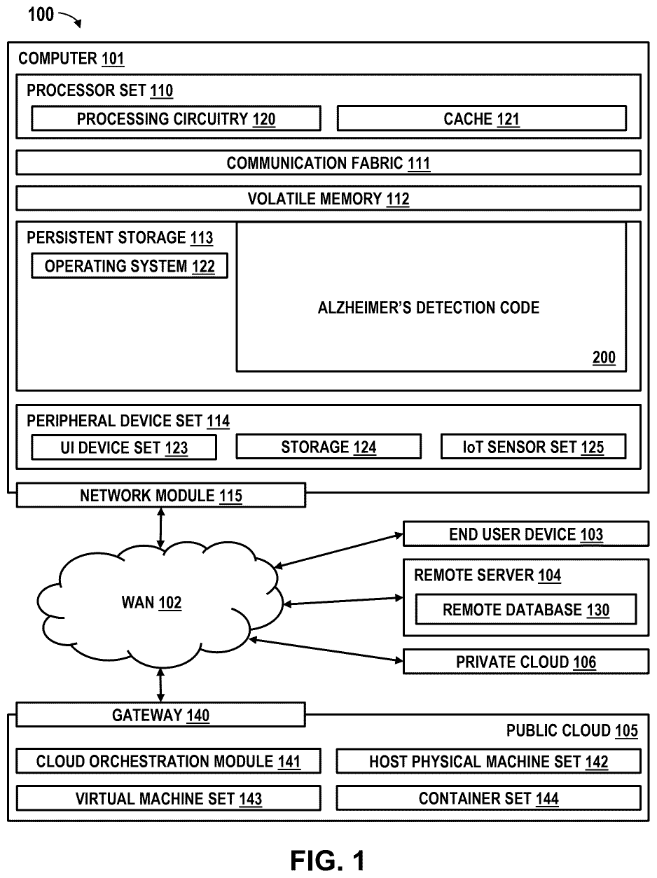

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer-readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer-readable program instructions are stored in various types of computer-readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the Internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the Internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer-readable program instructions for performing the inventive methods can typically be downloaded to computer 101 from an external computer or external storage device through a network adapter card or network interface included in network module 115.

WAN 102 is any wide area network (for example, the Internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the Internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

CLOUD COMPUTING SERVICES AND/OR MICROSERVICES (not separately shown in FIG. 1): private and public clouds 106 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the Internet. Cloud services facilitate the flow of user data from front-end clients (for example, user-side servers, tablets, desktops, laptops), through the Internet, to the provider's systems, and back. In some embodiments, cloud services may be configured and orchestrated according to as “as a service” technology paradigm where something is being presented to an internal or external customer in the form of a cloud computing service. As-a-Service offerings typically provide endpoints with which various customers interface. These endpoints are typically based on a set of APIs. One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with these things. Another category is Software as a Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

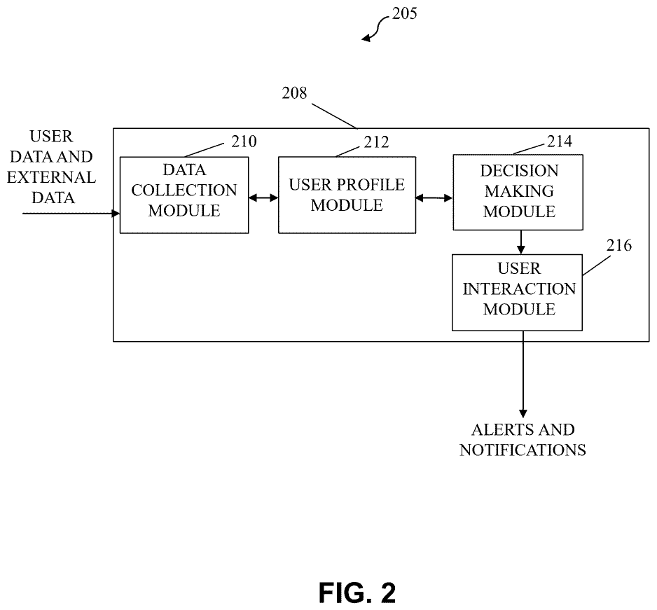

FIG. 2 shows a block diagram of an exemplary environment 205 in accordance with aspects of the present invention. In embodiments, the environment 205 includes a medical detection server 208, which may comprise one or more instances of the computer 101 of FIG. 1. In other examples, the medical detection server 208 comprises one or more virtual machines or one or more containers running on one or more instances of the computer 101 of FIG. 1.

In embodiments, the medical detection server 208 of FIG. 2 comprises a data collection module 210, a user profile module 212, a decision making module 214, and a user interaction module 216, each of which may comprise modules of the code of block 200 of FIG. 1. Such modules may include routines, programs, objects, components, logic, data structures, and so on that perform particular tasks or implement particular data types that the code of block 200 uses to carry out the functions and/or methodologies of embodiments of the present invention as described herein. These modules of the code of block 200 are executable by the processing circuitry 120 of FIG. 1 to perform the inventive methods as described herein. The medical detection server 208 may include additional or fewer modules than those shown in FIG. 2. In embodiments, separate modules may be integrated into a single module. Additionally, or alternatively, a single module may be implemented as multiple modules. Moreover, the quantity of devices and/or networks in the environment is not limited to what is shown in FIG. 2. In practice, the environment may include additional devices and/or networks; fewer devices and/or networks; different devices and/or networks; or differently arranged devices and/or networks than illustrated in FIG. 2.

In embodiments, the data collection module 210 receives user data from an Internet of things (IoT) network and/or at least one wearable device. In further embodiments, the IoT network monitors user interactions, actions, geolocation data, and task completions. In embodiments of the present invention, the IoT network integrates with at least one smart home appliance, at least one sensor, and at least one camera. In aspects of the present invention, the at least one wearable device collects physical data (e.g., blood pressure, blood temperature, and heartbeat), voice data (e.g., voice conversations, self talk, and user interactions), and daily activity data (e.g., movement data, positioning data, and use of objects).

In aspects of the present invention, the data collection module 210 receives user data which includes speech data, interaction data, and daily activity data. In further embodiments, the data collection module 210 receives external data from external data sources. In further embodiments, the speech data tracks changes in speech patterns over time, detecting slowness, repetition, or confusion, which are common symptoms of cognitive decline. In aspects of the present invention, the interaction data monitors social engagement, response times, and the nature of communications (e.g., confused, hesitant, or misinterpreted responses). In embodiments of the present invention, the daily activity data analyzes the completion of familiar tasks (e.g., cooking, dressing, etc.) and detects any unusual behavior (e.g., forgetting steps, using items incorrectly, etc.). In aspects of the present invention, the external data sources include calendar events, task lists, and reminders to assess whether the user can still manage daily responsibilities and plan effectively. In further embodiments, the data collection module 210 pre-processes the user data by normalizing numerical data (e.g., scaling heart rate, blood pressure, etc.). In aspects of the present invention, the data collection module 210 also pre-processes the user data by using one-hot encoding or label encoding for categorical data, such as interactions with predetermined objects or activities. In further embodiments of the present invention, the data collection module 210 further pre-processes the data by dropping information which corresponds with missing data points or inputting missing data points. The data collection module 210 sends the user data to the user profile module 212.

In aspects of the present invention, the user profile module 212 receives the user data from the data collection module 210 and creates a user profile by using a generative artificial intelligence (AI) model which continuously gathers and analyzes the user data from the IoT network and/or at least one wearable device. In particular, the user profile module 212 creates the user profile which comprises profile elements including speech patterns, interaction habits, emotional preferences, task completion, abnormal vital signs, motion patterns, unusual geospatial movement, and interactions with objects. In embodiments, the speech patterns comprise tone, speed, speech clarity, speech rate, sentiment, and complexity of spoken language. In further embodiments, the interaction habits comprise frequency and type of interactions with others, responsiveness, and engagement levels (e.g., whether the user has conversational difficulty). In embodiments, the emotional preferences comprise facial expressions from video, voice sentiments, interactions, and identifying emotional states and variations (e.g., whether the user has more confused speech). In aspects of the present invention, the task completion comprises the ability to complete tasks (e.g., whether the user is forgetting familiar tasks). In embodiments, the abnormal vital signs comprise heart rate, blood pressure trends, etc. In further embodiments, the motion patterns of the user include irregular walking or inactivity. In aspects of the present invention, the unusual geospatial movement comprises wandering beyond safe zones. In embodiments of the present invention, the interaction with objects comprise dangerous actions like approaching stoves or using knives. In aspects of the present invention, the user profile module 212 determines the user profile which comprises the speech patterns, the interaction habits, the emotional preferences, task completion, abnormal vital signs, motion patterns, unusual geospatial movement, and interactions with objects based on the user data. The user profile module 212 sends the user profile which comprises the speech patterns, the interaction habits, emotional preferences, task completion, abnormal vital signs, motion patterns, unusual geospatial movement, and interactions with objects to the decision making module 214.

In embodiments of the present invention, the decision making module 214 receives the user profile. In further embodiments, the decision making module 214 builds and trains an artificial intelligence (AI) model by utilizing a random forest algorithm. In embodiments, the AI model is trained using the user profile. In further embodiments, the AI model is also trained using historical user profiles which comprises historical speech patterns, historical interaction habits, historical emotional preferences of patients which have been previously diagnosed with Alzheimer's. In aspects of the present invention, the decision making module 214 builds and trains the AI model by utilizing the random forest algorithm. In embodiments, the random forest algorithm creates multiple decision trees and splits the dataset for each decision tree based on feature values to maximize information gain and reduce impurity. Further, the random forest algorithm trains each decision tree using a random subset of features and random subsets of data points to ensure that the decision trees are diverse. In aspects of the present invention, the random forest algorithm provides a measure of feature importance. In embodiments, feature importance corresponds with how much a particular feature contributes to the prediction of Alzheimer risk. In particular, feature importance determines which data points are most indicative of Alzheimer's risk. In an example, a speech rate might have feature importance which is more important than physical data.

In aspects of the present invention, the decision making module 214 utilizes the AI model to output the binary classification. In embodiments, the decision making module 214 utilizes the AI model to output the binary classification which is a prediction of whether the user is at a high risk for Alzheimer's (e.g., binary classification=1) or at a low risk for Alzheimer's (e.g., binary classification=0). In aspects of the present invention, the AI model determines whether there is a high risk for Alzheimer's by comparing the current user profile against historical profiles to output the binary classification that is either 1 for a high risk of Alzheimer's or 0 for a low risk of Alzheimer's. In contrast to a cognitive state score (CSS) and real-time CSS described below, the decision making module 214 determines the binary classification without incorporating a time element or time period.

In further embodiments, the decision making module 214 utilizes the AI model to determine the Alzheimer's risk score based on a cognitive state score (CSS). In aspects of the present invention, the CSS measures cognitive changes over a predetermined period (e.g., weekly, monthly, etc.). In other words, the CSS comprises a long term CSS over the predetermined period of time. In aspects of the present invention, the AI model detects patterns to indicate cognitive decline by comparing the current user profile against historical profiles and outputs the CSS that reflects a cognitive health trajectory of a user. For example, a decreasing CSS over time signals a potential for the user to have Alzheimer's. In this situation, the decreasing CSS over time can prompt further investigation by a caregiver, a family member, or the user.

In further embodiments of the present invention, the decision making module 214 utilizes the AI model to determine the Alzheimer's risk score based on a real-time CSS. In aspects of the present invention, the real-time CSS provides an immediate assessment of a cognitive state of the user at any given moment in time. For example, the real-time CSS provides the immediate assessment of the cognitive state of the user at 2:03 PM EST on Tuesday (i.e., the given moment), while the CC provides the cognitive state of the user over an entire week (i.e., over a predetermined period). In this scenario, the decision making module 214 utilizes the AI model to continuously update the real-time CSS as new data is received. In this scenario, the decision making module 212 allows for dynamic and on-the-fly adjustments to an environment of the user based on the real-time CSS. For example, the decision making module 212 utilizes the real-time CSS for an immediate protective action, such as informing a caregiver or a family member to restrict the user to certain areas. In another example, the decision making module 212 utilizes the real-time CSS for another immediate protective action, such as sending an alert to a caregiver or family member in response to the real-time CSS suddenly dropping quickly (e.g., the real-time CSS dropping over a predetermined amount within a predetermined time period).

In aspects of the present invention, the decision making module 214 utilizes the AI model to determine the CSS as follows:

$$CSS = ( 1 / N ) * ( {\sum }_{i = 1}^{N} ( Wi * fi ) ) . \qquad ( Equation 1 )$$

In Equation 1, N represents a total number of cognitive factors being assessed (e.g., speech, memory, task completion, spatial awareness, etc.), Wi is a weight assigned to each factor i and reflects an importance of each factor in an overall assessment of the user, fi is a normalized score for each factor i based on a performance of the user. In particular, the normalized score of 0 represents severe cognitive impairment and 1 represents normal cognitive functions. In embodiments, the CSS is determined by utilizing Equation 1 over a predetermined period of time (i.e., a week). In further embodiments, the real-time CSS is determined by utilizing Equation 1 over a given moment (i.e., 2:03 PM EST on a Tuesday).

In further embodiments, factors can comprise a speech pattern score S, a memory recall score M, a task completion score T, a spatial awareness score P, etc. In aspects of the present invention, the speech pattern score S is assessed through the AI model analyzing speech data of the user (e.g., clarity, coherence, hesitation, etc.). In embodiments of the present invention, the memory recall score M is assessed through the AI model analyzing the ability of the user to remember tasks, events, action completion, and/or interactions. In further embodiments of the present invention, the task completion score T is assessed through the AI model evaluating how well the user completes daily tasks (e.g., dressing, cooking, etc.) In aspects of the present invention, the spatial awareness score P is assessed through the AI model determining how well a user understands an environment, such as recognizing familiar places or being able to navigate a home of the user. In further embodiments of the present invention, the decision making module 212 assigns a different weight Wi to each factor depending on the importance of the factor. For example, Ws (i.e., a weight of the speech pattern score Ws) is higher than Wt (i.e., a weight of the task completion score Wt) in response to a determination that speech patterns are more critical than task completions.

In aspects of the present invention, the decision making module 214 evaluates the AI model by using performance metrics such as accuracy, precision and recall, an F1 score, and a receiver operating characteristic-area under the curve (ROC-AUC). In embodiments, accuracy comprises a measure of how often the AI model predicts the Alzheimer's risk score and the binary classification correctly. In further embodiments, precision comprises relevant retrieved instances divided by all retrieved instances and recall comprises relevant retrieved instances divided by all relevant instances. In aspects of the present invention, precision and recall is important if the dataset is imbalanced (i.e., more non-Alzheimer's cases than Alzheimer's cases). In embodiments of the present invention, the F1 score comprises a harmonic mean of precision and recall. In aspects of the present invention, the ROC-AUC evaluates a trade-off between a true positive rate and a false positive rate.

In embodiments of the present invention, the decision making module 214 utilizes the AI model to determine the Alzheimer's risk score based on the CSS and the real-time CSS. In further embodiments, the decision making module 214 uses the CSS and the real-time CSS to predict a likelihood of the onset of Alzheimer's for the user. In this situation, the decision making module 214 sends the prediction of the likelihood of the onset of Alzheimer's for the user (i.e., the Alzheimer's risk score), the binary classification, the CSS, and the real-time CSS to the user interaction module 216.

In aspects of the present invention, the user interaction module 216 receives the prediction of the likelihood of the onset of Alzheimer's for the user (i.e., the Alzheimer's risk score) from the decision making module 214. In embodiments, the user interaction module 216 automatically notifies a family member, the user, or a caregiver to seek medical tests in response to the Alzheimer's risk score being less than a predetermined Alzheimer's threshold. In this scenario, the Alzheimer's risk score being less than the predetermined Alzheimer's threshold represents a high likelihood that that user has Alzheimer's. In further embodiments, the user interaction module 216 provides a trend visualization of cognitive health of the Alzheimer's risk score over time through an interactive and intuitive graphical user interface (GUI). In further aspects of the present invention, the user interaction module 216 outputs the trend visualization to a mobile device of the user, the family member, or the caregiver so that the user, the family member, or the caregiver can view the data trends over time of the Alzheimer's risk score. Accordingly, by tracking the data trends over time of the Alzheimer's risk score, the user, the family, or the caregiver can intervene early and provide proactive protections to the user.

In further embodiments of the present invention, the user interaction module 216 receives the binary classification from the decision making module 214. In aspects of the present invention, the user interaction module 216 automatically notifies a family member, the user, or a caregiver to seek medical tests in response to the binary classification indicating a high risk of Alzheimer's (e.g., binary classification=1). In further embodiments, the user interaction module 216 provides the trend visualization of the binary classification over time through the interactive and intuitive GUI.

In embodiments of the present invention, the user interaction module 216 uses the real-time CSS to output a first geofence notification which dynamically adjusts a geofence according to a plurality of predefined policies to protect the user. In this scenario, the user interaction module 216 outputs the first geofence notification in response to the real-time CSS being below a real-time CSS threshold. For example, the real-time CSS being below the real-time CSS threshold represents a high likelihood that the cognitive state of the user has dropped suddenly and indicates that the user has Alzheimer's. Accordingly, the user needs dynamic adjusting of geofencing to ensure user safety in response to a sudden change in the real-time CSS. In other embodiments, the dynamic adjusting of geofencing occurs in response to the Alzheimer's risk score being below the predetermined Alzheimer's threshold.

In further embodiments of the present invention, the user interaction module 216 uses the real-time CSS to recognize a risky object within a vicinity of the user or risk actions of the user. For example, the user interaction module 216 uses the real-time CSS to set up object recognition to determine that the user is using a dangerous object (e.g., a knife). In this scenario, the user interaction module 216 activates an accessible mode on a wearable device of the user to guide the user either through audible instructions or visual instructions so that the user does not get harmed while using the knife. In another example, the user interaction module 216 uses the real-time CSS to set up action recognition to determine that the user is performing a risky action (e.g., using a gas stove). In this scenario, the user interaction module 216 activates another accessible mode on the wearable device of the user to guide the user either through audible instructions or visual instructions so that the user moves away from the gas stove and prevents the user from being harmed by the gas stove.

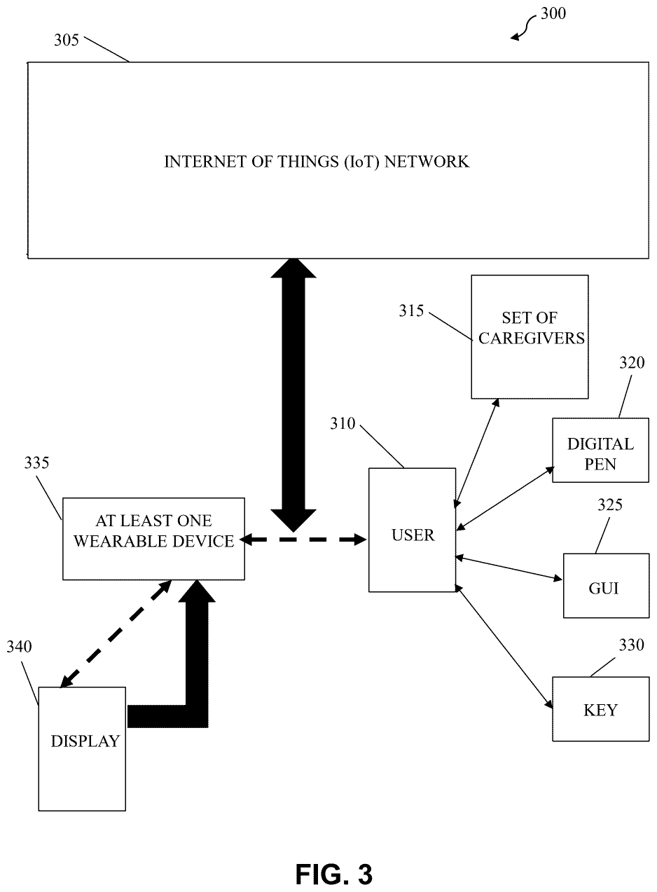

FIG. 3 shows a block diagram of a data collection system in accordance with aspects of the present invention. In FIG. 3, the data collection system 300 comprises an Internet of things (IoT) network 305, a user 310, a set of caregivers 315, a digital pen 320, a graphical user interface (GUI) 325, a key 330, at least one wearable device 335, and a display 340. In FIG. 3 the IoT network 305 comprises a camera, a geolocation device, a voice recorder, and a geofenced environment. In FIG. 3, the user 310 wears at least one wearable device 335 with the display 340. In further embodiments, the set of caregivers 315 utilize the digital pen 320 with the GUI 325 to track a progress of the user 310 and ensure that the user 310 does not have access to the key 330 to leave the geofenced environment.

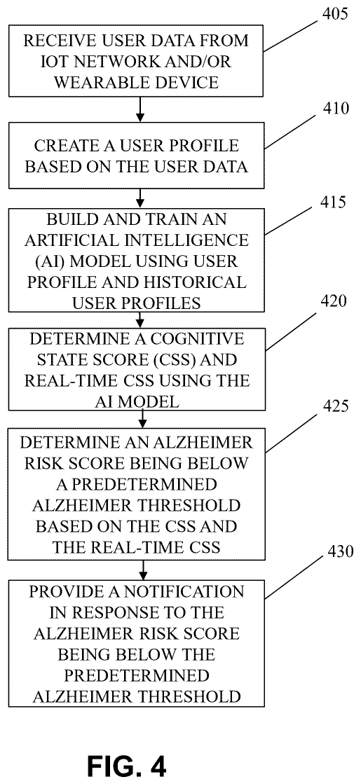

FIG. 4 shows a flowchart of an exemplary method in accordance with aspects of the present invention. Steps of the method may be carried out as operations in the environment 205 of FIG. 2 and are described with reference to elements depicted in FIG. 2.

At step 405, the system receives, at the data collection module 210, user data from an Internet of things (IoT) network and/or at least one wearable device. In embodiments and as described with FIG. 2, the user data comprises speech data, interaction data, daily activity data, and external data sources. At step 410, the system creates, at the user profile module 212, the user profile based on the user data. In embodiments and as described with FIG. 2, the user profile comprises speech patterns, interaction habits, emotional preferences, task completion, abnormal vital signs, motion patterns, unusual geospatial movement, and interactions with objects. In embodiments, the speech patterns comprise tone, speed, speech clarity, speech rate, sentiment, and complexity of spoken language.

At step 415, the system builds and trains, at the decision making module 214, an artificial intelligence (AI) model using the user profile and historical user profiles. In embodiments and as described with FIG. 2, the AI model is trained using a random forest algorithm. At step 420, the system determines, at the decision making module 214, a cognitive state score (CSS) and real-time CSS using the AI model. In embodiments and as described with FIG. 2, the CSS represents a cognitive state over a period of time and the real-time CSS represents the cognitive state at a current period of time.

At step 425, the system determines, at the decision making module 214, an Alzheimer's risk score being below a predetermined Alzheimer's threshold based on the CSS and the real-time CSS. In embodiments and as described with FIG. 2, the Alzheimer's risk score comprises a prediction of the likelihood of the onset of Alzheimer's for the user. At step 430, the system provides, at the user interaction module 216, a notification in response to the Alzheimer's risk score being below the predetermined Alzheimer's threshold. In embodiments and as described in FIG. 2, the Alzheimer's risk score being less than the predetermined Alzheimer's threshold represents a high likelihood that that user has Alzheimer's. In further embodiments and as described with FIG. 2, the notification is provided to at least one of a caregiver, a family member, or the user.

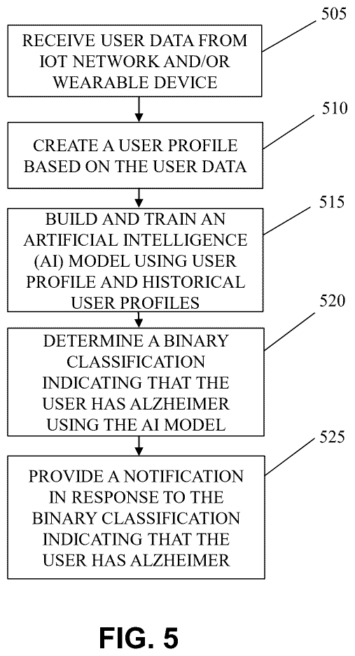

FIG. 5 shows a flowchart of an exemplary method in accordance with aspects of the present invention. Steps of the method may be carried out as operations in the environment 205 of FIG. 2 and are described with reference to elements depicted in FIG. 2.

At step 505, the system receives, at the data collection module 210, user data from an Internet of things (IoT) network and/or at least one wearable device. In embodiments and as described with FIG. 2, the user data comprises speech data, interaction data, daily activity data, and external data sources. At step 510, the system creates, at the user profile module 212, the user profile based on the user data. In embodiments and as described with FIG. 2, the user profile comprises speech patterns, interaction habits, emotional preferences, task completion, abnormal vital signs, motion patterns, unusual geospatial movement, and interactions with objects. In embodiments, the speech patterns comprise tone, speed, speech clarity, speech rate, sentiment, and complexity of spoken language.

At step 515, the system builds and trains, at the decision making module 214, an artificial intelligence (AI) model using the user profile and historical user profiles. In embodiments and as described with FIG. 2, the AI model is trained using a random forest algorithm. At step 520, the system determines, at the decision making module 214, a binary classification which indicates that the user is at a high risk for Alzheimer's using the AI model.

At step 525, the system provides, at the user interaction module 216, a notification in response to the binary classification which indicates that the user is at a high risk for Alzheimer's using the AI model. In embodiments and as described with FIG. 2, the notification is provided to at least one of a caregiver, a family member, or the user.

In still additional embodiments, the present invention provides a computer-implemented method, via a network. In this case, a computer infrastructure, such as computer 101 of FIG. 1, can be provided and one or more systems for performing the processes of the present invention can be obtained (e.g., created, purchased, used, modified, etc.) and deployed to the computer infrastructure. To this extent, the deployment of a system can comprise one or more of: (1) installing program code on a computing device, such as computer 101 of FIG. 1, from a computer readable medium; (2) adding one or more computing devices to the computer infrastructure; and (3) incorporating and/or modifying one or more existing systems of the computer infrastructure to enable the computer infrastructure to perform the processes of the present invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
receiving user data from a device;
creating a user profile based on the user data;
training an artificial intelligence (AI) model using the user profile and historical user profiles;
determining a long term cognitive state score (CSS) and a real-time CSS using the trained AI model;
determining that an Alzheimer's risk score is below a predetermined Alzheimer's threshold based on the long term CSS and the real-time CSS; and
providing a notification in response to the Alzheimer risk score being below the predetermined Alzheimer's threshold.

2. The method of claim 1, wherein:
the creating the user profile based on the user data further comprises creating the user profile based on the user data by utilizing a generative AI model; and
the device comprises at least one wearable device.

3. The method of claim 1, wherein the user profile comprises speech patterns, interaction habits, emotional preferences, task completion, abnormal vital signs, motion patterns, unusual geospatial movements, and interactions with objects.

4. The method of claim 1, wherein the AI model is trained using a random forest algorithm.

5. The method of claim 1, wherein the long term CSS represents a cognitive state over a predetermined period of time.

6. The method of claim 5, wherein the long term CSS is determined based on plural cognitive factors, a weight assigned to each of the cognitive factors, and a normalized score for each of the cognitive factors.

7. The method of claim 6, wherein the long term CSS is determined by a formula including: long term
$$CSS = ( 1 / N ) * ( {\sum }_{i = 1}^{N} ( Wi * fi ) ) ,$$
in which N is the number of cognitive factors, Wi is the weight assigned to each cognitive factor, and fi is the normalized score for each cognitive factor.

8. The method of claim 1, wherein the Alzheimer's risk score comprises a prediction of a likelihood of an onset of Alzheimer's.

9. The method of claim 1, wherein the notification is provided to a caregiver.

10. The method of claim 9, wherein the notification provided to the caregiver comprises a geofence notification which dynamically adjusts a geofence according to a plurality of predefined policies.

11. The method of claim 1, further comprising providing a trend visualization of cognitive health of the Alzheimer's risk score over time through an interactive graphical user interface (GUI).

12. The method of claim 1, further comprising:
determining that a user associated with the user data is using a dangerous object based on an object detection and the real-time CSS.

13. The method of claim 1, further comprising:
determining that a user associated with the user data is performing a risky action based on an action recognition and the real-time CSS.

14. A computer program product comprising:
one or more computer readable storage media; and
program instructions stored on the one or more computer readable storage media to perform operations comprising:
receiving user data from an Internet of things (IoT) network;
creating a user profile based on the user data;
training an artificial intelligence (AI) model using the user profile and historical user profiles by utilizing a random forest algorithm;
determining a long term cognitive state score (CSS) and a real-time CSS using the trained AI model;
determining that an Alzheimer's risk score is below a predetermined Alzheimer's threshold based on the long term CSS and the real-time CSS; and
providing a notification in response to the Alzheimer's risk score being below the predetermined Alzheimer's threshold.

15. The computer program product of claim 14, wherein the user profile comprises speech patterns, interaction habits, emotional preferences, task completion, abnormal vital signs, motion patterns, unusual geospatial movements, and interactions with objects.

16. The computer program product of claim 14, wherein the long term CSS represents a cognitive state over a predetermined period of time.

17. The computer program product of claim 16, wherein the long term CSS is determined based on plural cognitive factors, a weight assigned to each of the cognitive factors, and a normalized score for each of the cognitive factors.

18. The computer program product of claim 17, wherein the long term CSS is determined by a formula including: long term
$$CSS = ( 1 / N ) * ( {\sum }_{i = 1}^{N} ( Wi * fi ) ) ,$$
in which N is the number of cognitive factors, Wi is the weight assigned to each cognitive factor, and fi is the normalized score for each cognitive factor.

19. The computer program product of claim 14, wherein the Alzheimer's risk score comprises a prediction of a likelihood of an onset of Alzheimer's.

20. A system comprising:
a processor set;
one or more computer readable storage media; and
program instructions stored on the one or more computer readable storage media to cause the processor set to perform operations comprising:
receiving user data from a wearable device;
creating a user profile based on the user data;
training an artificial intelligence (AI) model using the user profile and historical user profiles by utilizing a random forest algorithm;
determining a long term cognitive state score (CSS) and a real-time CSS using the trained AI model;
determining that an Alzheimer's risk score is below a predetermined Alzheimer's threshold based on the long term CSS and the real-time CSS;
providing a caregiver notification in response to the Alzheimer's risk score being below the predetermined Alzheimer's threshold; and
providing a geofence notification which dynamically adjusts a geofence for protection in response to the real-time CSS being below a real-time CSS threshold.
